(19) United States
(12) Patent Application Publication
(10) Pub. No.: US 2014/0194392 A1
(43) Pub. Date: Jul. 10, 2014

(54) DRUG SCREENING METHOD

(71) Applicant: NATIONAL CHIAO TUNG UNIVERSITY, Hsinchu (TW)

(72) Inventors: Jinn-Moon Yang, Hsinchu (TW); Kai-Cheng Hsu, Hsinchu (TW)

(73) Assignee: NATIONAL CHIAO TUNG UNIVERSITY, Hsinchu (TW)

(21) Appl. No.: 13/872,456

(22) Filed: Apr. 29, 2013

(30) Foreign Application Priority Data
Jan. 9, 2013 (TW) 102100677

(51) Int. Cl. G01N 33/573; A61K 31/255; A61K 31/196; A61K 31/662; A61K 31/185

(52) U.S. Cl. CPC G01N 33/573; A61K 31/662; A61K 31/185; A61K 31/196; A61K 31/255
USPC 514/114; 506/9; 514/577; 514/562; 514/510

(57) ABSTRACT

A drug screening method is disclosed. The drug screening method includes steps of screening a compound library to obtain a first group of compounds capable of binding to a wild type target; screening the first group of compounds to obtain a second group of compounds capable of binding to a mutant site of a mutant target; analyzing characteristics of binding sites of the wild type target and the mutant type target to obtain physico-chemical properties of the binding sites; identifying a candidate from the second group of compounds according to the physico-chemical properties of the binding site; and performing a bio-assay on inhibitory activity of the candidate.

### BACKGROUND OF THE INVENTION

1. Field of the Invention

The present application relates to a drug screening method, and more particularly, to a drug screening method for both wild type and mutant type targets.

2. Description of Related Art

The influenza virus is simply known as the flu virus, and is a type of ribonucleic acid (RNA) virus that can cause acute respiratory tract infections in humans or animals. In taxonomical studies, the influenza virus is a virus of the Orthomyxoviridae family; and according to the sequences of nucleoprotein and matrix protein, it can be classified into 3 serotypes: influenza A, influenza B and influenza C, wherein influenza A has the highest morbidity. The swine influenza caused by the swine-origin influenza A (H1N1) virus has become a world epidemic in April 2009. These mutated human influenza virus, swine influenza virus and bird influenza are still common nowadays.

Neuraminidase (NA) is a type of glycoprotein located on the surface of the influenza virus envelope. NA plays a role in helping mature virus particles leave the host cell during the process of viral replication, to infect new cells. Therefore, NA plays an essential role in the spread of the influenza virus, and as such it has become one of the important targets in the development of drugs to combat influenza. The current clinical drugs used to combat influenza include zanamivir (Relenza®) and oseltamivir (Tamiflu®), and these belong to inhibitors of NA, such that these drugs can be used to prevent the spread of virus, thus achieving the effects of combating influenza. However, zanamivir and oseltamivir are expensive. Furthermore, since the influenza virus has the nature to mutate easily, there are already known cases of drug resistance against zanamivir and oseltamivir. There are even cases of mutant type virus strains which have multiple drug resistances. Some mutations of NA alter the binding site characteristics, and even disrupt the binding of drugs. For example, the H275Y mutation is a mutation associated with a common drug resistance. The H275Y mutation significantly reduces the activity of GS4071. It is known that the I223R and H275Y double mutation reduces the activity of zanamivir by approximately 20 folds, and this mutation also reduces the activity of GS4071 by approximately 29,915 folds.

Therefore, there is a need to search for compounds capable of simultaneously inhibiting the wild type target and mutant type (resistant) targets through an effective drug screening method.

### SUMMARY OF THE INVENTION

A drug screening method is provided. The drug screening method includes steps of screening a compound library to obtain a first group of compounds capable of binding to a wild type target; screening the first group of compounds to obtain a second group of compounds capable of binding to a mutant site of a mutant target; analyzing characteristics of binding sites of the wild type target and the mutant type target to obtain physico-chemical properties of the binding sites; identifying a candidate from the second group of compounds according to the physico-chemical properties of the binding site; and performing a bio-assay on inhibitory activity of the candidate.

According to an embodiment, the first group of compounds are screened out from the compound library by a molecular docking tool. According to an embodiment, the second group of compounds are screened out from the first group of compounds by the molecular docking tool.

According to an embodiment, the candidate is capable of binding to the wild type target and the mutant type target. In one embodiment, the candidate has inhibitory potential on the wild type target and the mutant type target.

According to an embodiment, docking models of the second group of compounds and the binding sites are used for analyzing the characteristics of the binding sites. According to an embodiment, the analysis of the binding site characteristics is demonstrated by site-moiety map. According to an embodiment, docking models of the second group of compounds and the binding sites are used for analyzing the characteristics of the binding sites and establishing the site moiety map. In one embodiment, docking models of the second group of compounds and the binding sites are obtained by the molecule docking tool, and the characteristics of the binding sites are analyzed according the site moiety map.

According to an embodiment, the wild type target and the mutant type target are enzymes. In one embodiment, the mutation site of the mutant type target is located at the binding site of the mutant target. In one embodiment, the wild type target and the mutant type target are NAs (neuraminidases). In an embodiment, the mutant type target is mutant type NA, and the mutant site of mutant type NA is located at its binding site. According to an embodiment, the wild type target and the mutant type target are NAs, and the candidate is capable of inhibiting NA. In an embodiment, the candidate is used for treating NA related diseases or disorders.

The drug screening method of the present invention can effectively obtain compounds capable of simultaneously inhibiting wild type target and mutant type target with drug-resistance.

In one embodiment, a compound of formula (I) is used for manufacturing an inhibitor of NA

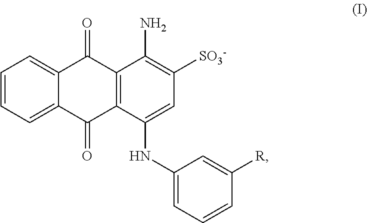

wherein R is one selected from the group consisting of:

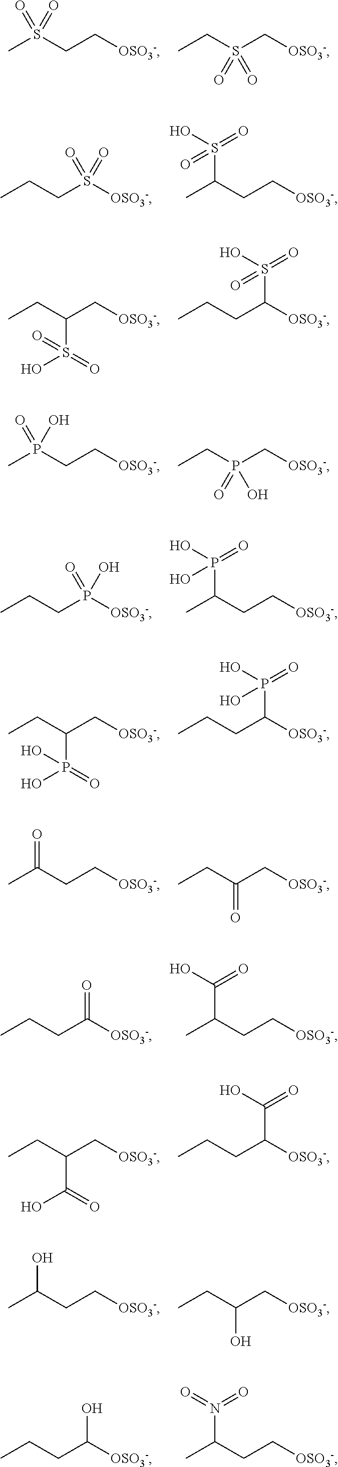

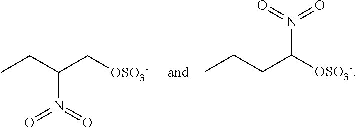

In one embodiment, R is

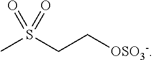

According to an embodiment, the NA comprises wild type NA and mutant type NA.

In one embodiment, a compound of formula (I) is used for manufacturing drugs for treating influenza:

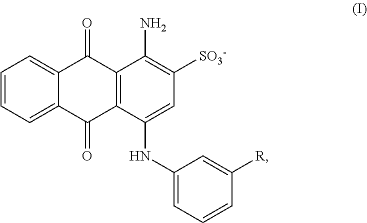

wherein in the formula, R is one selected from the group consisting of:

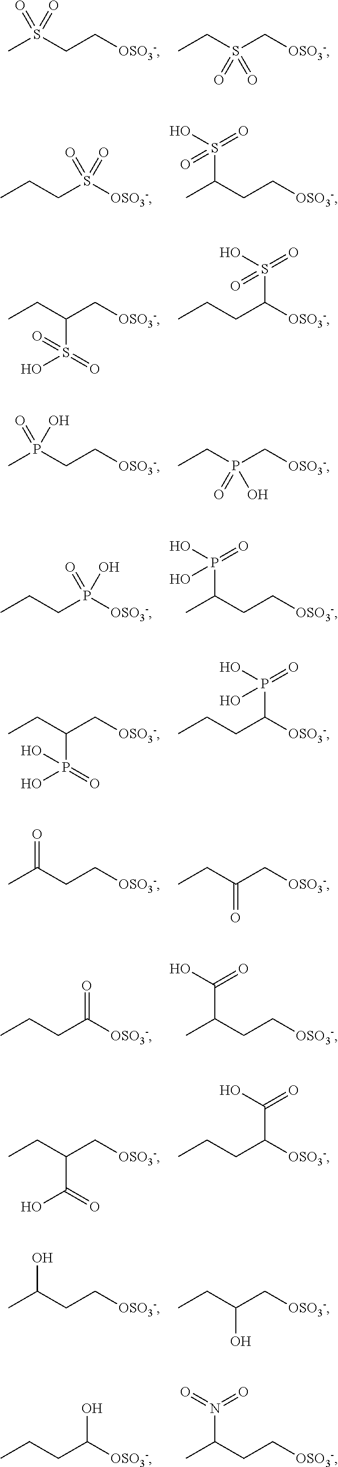

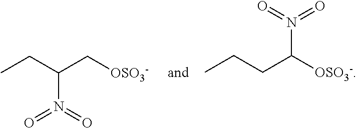

In one embodiment, R is

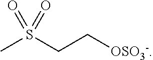

According to an embodiment, the NA comprises wild type NA and mutant type NA.

A method of inhibiting NA is provided. The method includes a step of administering to a subject a therapeutically effective amount of a compound of formula (I)

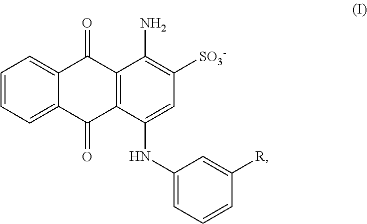

wherein R is one selected from the group consisting of:

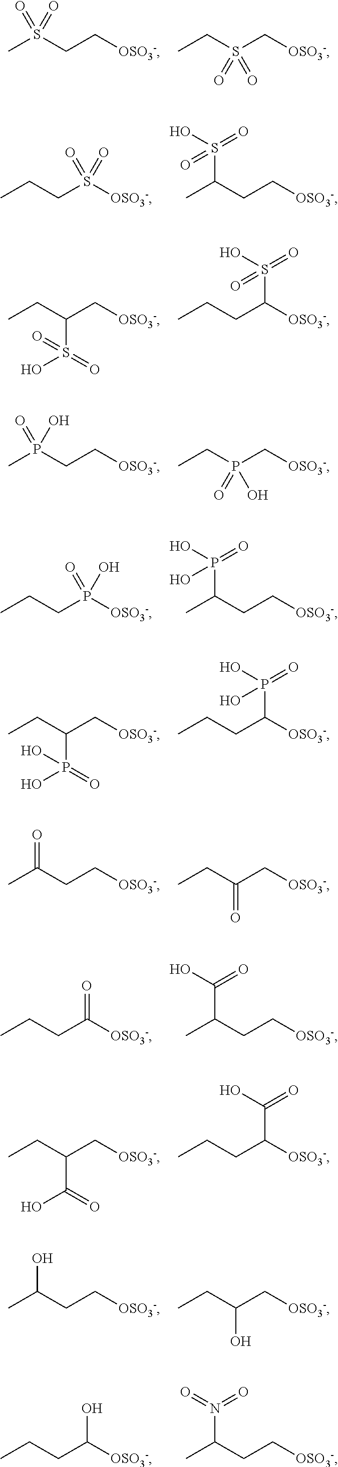

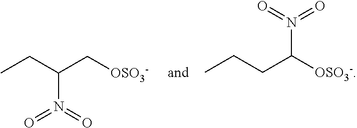

In one embodiment, R is

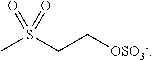

According to an embodiment, the NA comprises wild type NA and mutant type NA.

A method of treating influenza is provided. The method includes a step of administering to a subject a therapeutically effective amount of a compound of formula (I)

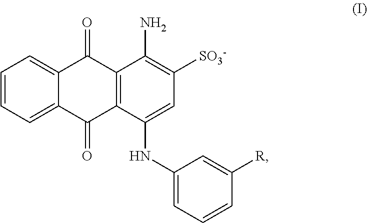

wherein R is one selected from the group consisting of:

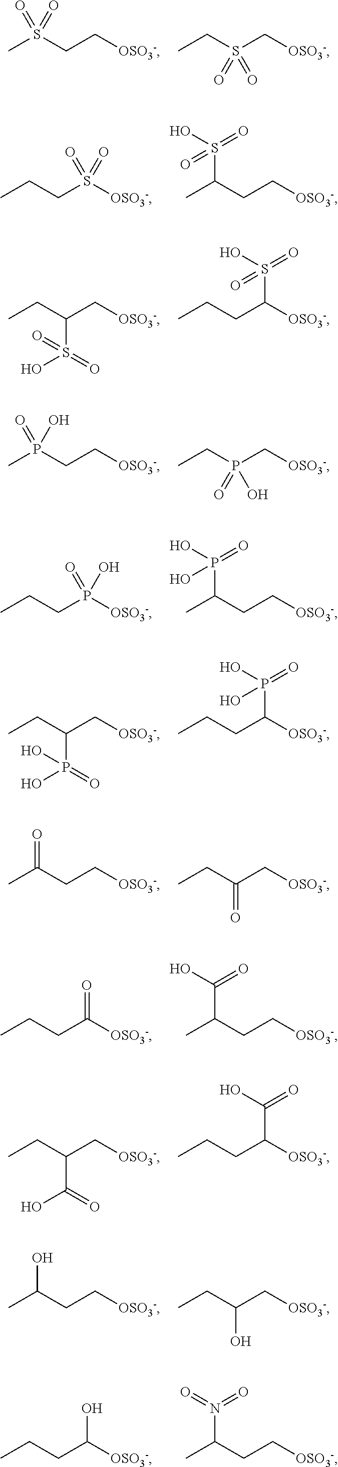

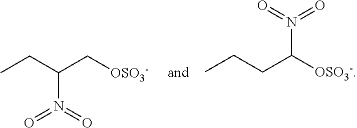

In one embodiment, R is

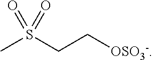

According to an embodiment, the compound of formula (I) has inhibitory activity on NA. In one embodiment, the NA comprises wild type NA and mutant type NA.

### BRIEF DESCRIPTION OF THE DRAWINGS

The Tables of the specification and drawings have the following abbreviation: neuraminidase is abbreviated as NA; wild type is abbreviated as WT; and multiple drug-resistance is abbreviated as MDR.

FIG. 1A to 1D show a flowchart of the parallel screening strategy in accordance with an embodiment of this invention. (A) Parallel screening for inhibitors of WT and MDR NAs. Compounds of the compound library were docked into both WT and MDR NAs using GEMDOCK. (B) Characterization of the mutant subsite by site-moiety map analysis. In the NA site-moiety map, the hydrogen-bonding anchor (colored green) represents a polar environment interacting with polar moieties. (C) Selection of potential anti-resistance inhibitors. Compounds that simultaneously matched characteristics of the 5 subsites for the WT and MDR NAs were selected. (D) Bioassay for verifying the effects of selected compounds on WT and MDR NAs.

FIG. 2A to 2D show the characteristic comparisons between WT and MDR NAs according to an embodiment of the present invention. (A) Binding site residues of MDR NAs and (B) WT NAs. The binding site was divided into the 5 subsites S1 (R118, R293, and R368), S2 (E119, D151, W179, and E228), S3 (R152, W179, and I223), S4 (I223, R225, and S247), and S5 (S247 and E277). The negative/positive, polar, hydrophobic, and mixed hydrophobic and polar subsites are shown as red, green, gray, and orange curves, respectively. These residues are shown in N1 numbering. Molecular surfaces represented by electrostatic potentials of (C) WT and (D) MDR NAs. The negative, positive, and neutral/hydrophobic potentials are colored red, blue, and white, respectively.

FIGS. 3A and 3B show the residues, moiety preferences, and interaction types of anchors according to an embodiment of the present invention. Residues, moiety preferences, and interaction types of anchors in (A) the mutant subsite and (B) the WT subsite. Anchors contain conserved interacting residues, moiety preferences, and interaction types. The hydrogen-bonding anchor (green) indicates that the mutant subsite is polar and prefers to form hydrogen bonds with polar moieties. In contrast, the WT subsite has a van der Waals anchor (gray).

FIGS. 4A to 4D show a comparison of binding conformations of RB19, zanamivir and GS4071 according to an embodiment of the present invention. (A) Docking conformation of RB19 on WT NA with hydrogen-bonding interactions is represented as dotted lines. (B) Conformations of GS4071 (black) and zanamivir (white) in WT NAs (lighter shade of white color) and MDR (dark grey) NAs. The GS4071 and zanamivir conformations were derived by superimposing N1 crystal structures (PDB code 3B7E and 2HU4). (C) Fold changes in IC₅₀ of RB19, zanamivir, and GS4071 when the dual H275Y/I223R mutation arises. (D) Docked conformation of RB19 on MDR NA.

FIG. 5 shows the interaction preference of mutant site according to an embodiment of the present invention. Protein-compound interaction profiles. Unless the screening compound yielded interactions with the residues, otherwise the profile entry color becomes grey; conversely, the entry color is black. The frame shows the major difference of interaction preferences between WT and MDR NAs.

FIG. 6 shows inhibition of influenza infection and replication by RB19 in MDCK cells according to an embodiment of the present invention. (A) RB19 inhibited the influenza-induced cytopathic effect. In the antiviral neutralization test, MDCK cells were lysed 64 hours after A/WSN/33 infection, as shown in the VC (virus control) column RB19 was added to A/WSN/33-infected cells by two-fold serial dilution and starting with a concentration of 50 μM (left most column). (B) Inhibition of influenza virus plaque formation by RB19. Approximately 50-100 PFU/well of A/WSN/33 (H1N1) or A/Udorn/72 (H3N2) of influenza A virus was used to infect MDCK cells in 6-well plates. After the viral adsorption stage, 3 ml of agar was overlaid on media containing various concentrations of RB19. The concentration of RB19 is indicated at the top. (C) Reduction in viral yields from infected cells w/o RB19 at different concentrations. MDCK cells were infected with MOI 0.001 A/WSN/33 (H1N1), and various concentrations of RB19 were added at the adsorption stage of the A/WSN/33 replication cycle. At 48 hour post infection, culture supernatants were collected for virus titration to monitor the viral yield by means of NA activity.

### DETAILED DESCRIPTION OF THE PREFERRED EMBODIMENTS

Various specific details are provided herein to provide a more thorough understanding of the invention.

A drug screening method is provided. The drug screening method includes the steps of screening a compound library to obtain a first group of compounds capable of binding to a wild type target; screening the first group of compounds to obtain a second group of compounds capable of binding to a mutant site of a mutant target; analyzing characteristics of binding sites of the wild type target and the mutant type target to obtain physico-chemical properties of the binding sites; identifying a candidate from the second group of compounds according to the physico-chemical properties of the binding site; and performing a bio-assay on inhibitory activity of the candidate.

According to an embodiment, the compound library is screened by means of the molecular docking tool to obtain the first group of compounds having the potential to bind to wild type targets. According to an embodiment, the first group of compounds are screened by means of the molecular docking tool to obtain the second group of compounds having the potential to bind to the mutant sites of the mutant targets. According to an embodiment, the compound library is screened by means of the molecular docking tool to obtain the first group of compounds capable of binding to wild type targets. According to an embodiment, the first group of compounds are screened by means of the molecular docking tool to obtain the second group of compounds capable of binding to the mutant sites of the mutant targets.

The mutant site refers to a site where mutation occurs. According to an embodiment of the present invention, the mutant sites of the mutant targets are located within the binding sites of the mutant targets. According to an embodiment of the present invention, the binding site of a target contains one or more subsites; and the mutant site is a subsite where mutation occurs within a binding site. The mutant site contains different amino acids from the corresponding site of the wild type targets. In cases when the target is protein, for example (which may be, but not limited to, receptor, antibody, enzyme and the like), the mutations of some amino acids may affect the binding of the target and the ligand. For example, the mutation of certain amino acids at certain positions will affect the binding of the drug with the target, thus greatly increasing the occurrence of drug anti-resistance.

The kind of molecular docking tool (which is sometimes referred to as molecular docking program) is not particularly limited in the present invention. According to an embodiment, the compound library is screened by means of the molecular docking tool to obtain the first group of compounds capable of binding to wild type targets. According to an embodiment, the first group of compounds are screened by means of the molecular docking tool to obtain the second group of compounds capable of binding to the mutant sites of the mutant targets. Embodiments of the molecular docking tool include, but are not limited to, GEMDOCK, DOCK, FlexX, GOLD, ConsDock, AutoDock, GLIDE, ICM-Docking, CDOCKER, LibDock, LigandFit and the like. According to an embodiment, the molecular docking tool GEMDOCK (was developed by Professor Jinn-Moon Yang of National Chiao Tung University, Taiwan, and can be downloaded from the website http://gemdock.life.nctu.edu.tw/dock/download.php) is used. According to an embodiment, the first group of compounds capable of binding to the wild type targets are screened out from the compound library by means of GEMDOCK. According to an embodiment, the second group of compounds capable of binding to mutant sites of mutant targets are screened out from the first group of compounds by means of GEMDOCK.

The characteristic analysis (also referred as characterization) of the binding sites of both wild type targets and mutant type targets refers to obtaining the physico-chemical properties of the binding sites and other relevant information (for example, but not limited to, interactions of the binding sites such as electrostatic interactions, hydrogen bonds, van der walls interactions and the like). The characterization also aims to obtain information on the functional groups and residues related to the interactions, moiety preference of the binding sites, the shape or size of the binding site, and the like. According to an embodiment, the characterization of binding site of the wild type target and binding site of the mutant type is performed to obtain the physico-chemical properties and other relevant information on the binding sites.

The analysis of the physico-chemical properties of the binding sites of both wild type targets and mutant type targets may be further used for screening out candidates. According to an embodiment, the characteristics of the binding sites of both the wild type targets and mutant type targets are analyzed, so as to obtain physico-chemical properties of the wild type target binding site and mutant type target binding site, and then candidates are identified from the second group of compounds according to the physico-chemical properties of the binding sites of both the wild type targets and mutant type targets. The physico-chemical properties and the relevant information include, but are not limited to, functional groups, residue polarity, electrostatic forces of the residues, interactions of the residues, shapes of binding sites, sizes of binding sites and the like. For example, the analysis of the consensus interactions between the compounds and binding sites and other related information (such as the shape of the binding site space and the like) can be further used for screening out candidates. According to an embodiment, after screening out the second group of compounds from the first group of compounds, the interactions of the second group of compounds and the binding sites, as well as other related information such as the shape of the binding site space and the like, are analyzed, so as to further screen out candidates, wherein the binding sites include the. According to an embodiment, to achieve screening out drugs that are simultaneously effective against both the wild type and mutant type targets, the interactions of the compounds (for example, the second group of compounds) and the respective binding sites of wild type targets and mutant type targets and/or other related information (such as the shape of binding site space and the like) are analyzed, and the analysis results are further used for screening out the candidates.

The characterization of binding sites of the wild type target and/or the mutant type target can be analyzed and demonstrated by any appropriate means. Examples of the tools/programs used for characterization include, but are not limited to, site-moiety maps and the like. According to an embodiment, the characterization of the binding sites of the wild type targets and/or mutant type targets is performed and demonstrated by the site-moiety map (SiMMap) (developed by Professor Jinn-Moon Yang of National Chiao Tung University, Taiwan, and can be downloaded from the website http://simfam.life.nctu.edu.tw/). The docking models of compounds and target binding sites and other relevant information can be used to assist the characterization. For instance, the docking models of the second group of compounds and the binding sites and relevant information can be used for characterization and establishment of the site-moiety map. The site-moiety map demonstrates the physico-chemical properties of the binding sites as well as other relevant information (such as, but are not limited to, interactions; the functional groups and residues involved in the interactions; the moiety preferences of the binding sites/binding subsites; the polarities of the residues, electrostatic interactions of the residues; shapes of the binding sites/binding subsites, sizes of the binding sites/binding subsites; and the like). The above-mentioned information can be used to further screen out candidates. According to an embodiment, the docking models of the second group of compounds obtained from the molecular docking tool and the binding sites (of wild type targets and mutant type targets) can be used to analyze the characteristics of the binding sites. In one embodiment, the docking models of the second group of compounds obtained from GEMDOCK and the binding sites (of wild type targets and mutant type targets) are used to analyze the characteristics of the binding sites. According to an embodiment, the characteristics analysis of the binding sites is demonstrated by the site-moiety map. In one embodiment, the docking models of the second group of compounds obtained from the molecular docking tool and the binding sites are used to analyze characteristics of the binding site, and to establish a site-moiety map. In one embodiment, the docking models of the second group of compounds obtained from the molecular docking tool and the binding sites, as well as the site-moiety map, are used to analyze characteristics of the binding site.

The candidates, which are screened out based on relevant information such as the physico-chemical properties of the binding sites have potential to bind to the wild type targets and mutant type targets. According to an embodiment, the candidates are screened out based on the physico-chemical properties of the binding sites, are capable of binding to the wild type targets and mutant type targets. According to an embodiment, the candidates have the potential to bind to the wild type targets and the mutant type targets; furthermore, the candidates have the potential to have interactions with the mutant sites of mutant targets. According to an embodiment, the candidates, which are screened out, are capable of binding to wild type targets and mutant type targets and have interactions with the mutant sites of mutant targets. According to an embodiment, the physico-chemical properties of candidates screened out from the second group of compounds are complementary to the physico-chemical properties of the binding sites (of wild type targets and mutant type targets. According to an embodiment, the candidates screened out from the second group of compounds have inhibitory potential on the wild type targets and the mutant type targets. According to an embodiment, the candidates screened out from the second group of compounds can inhibit the wild type targets and mutant type targets. According to an embodiment, candidates screened out from the second group f compounds can inhibit the wild type targets and mutant type targets, and also are capable of having interactions with the mutant sites of mutant targets.

Further bio-assays can be carried out to determine the inhibitory activity of the candidates after the screening out the candidates from the second group of compounds. According to an embodiment, bioassays of candidates are carried out to identify the drugs that have inhibitory activity on the wild type targets and mutant type targets. The bioassay method employed is not particularly limited, but must achieve determination of inhibitory activity.

The target in the screening method of the present invention may be any appropriate substance that can be used with such methods, for example, biological targets with binding sites, which may be, but not limited to proteins (for example, but are not limited to receptors, antibodys, enzymes and the like). According to an embodiment, the candidates that have inhibitory potential on the wild type targets and mutant type targets are screened out by the aforementioned drug screening method. According to an embodiment, drugs that can inhibit the wild type targets and mutant type targets are screened out by the aforementioned drug screening method. Certain mutations occurring at the binding sites of biological targets are related to drug resistance. According to an embodiment, candidates having inhibitory potential on wild type targets and mutant type targets are screened out by the aforementioned drug screening method. Therefore, candidates that can simultaneously inhibit wild type targets and those that have drug resistance (for example, pathogens) are screened out by the aforementioned drug screening method. According to an embodiment, drugs which can simultaneously inhibit wild type targets and mutant type targets are screened out by the drug screening method of the present invention. As such, the aforementioned drug screening method can screen out drugs which can inhibit wild type targets and those having drug resistance (such as pathogens), and can significantly benefit drug development.

The aforementioned drug screening method can be used to screen for prevention agents or treatment agents for various diseases or disorders. This drug screening method is particularly beneficial towards screening for prevention agents or treatment agents for diseases or disorders associated with genes having high mutation rates. The diseases or disorders include, but are not limited to, influenza, cancer, acquired immune deficiency syndrome (AIDS), tuberculosis, pneumonia and the like.

Candidates which have simultaneous inhibitory potential on wild type targets and mutant type targets are screened out by the drug screening method of the present invention. Drugs that have simultaneous inhibitory effects on both wild type targets and mutant type targets are screened out by the drug screening method of the present invention. As mentioned before, the occurrence of certain mutations leads to drug resistance. For instance, the I223R and H275Y double mutation of NA (neuraminidase) leads to drug resistance towards zanamivir and oseltamivir (the expression GS4071 may be used interchangeably with oseltamivir in the present invention). To illustrate this further, a clinical example whereby the occurrence of a drug-resistant mutation leads to malfunctioning of a drug is demonstrated by gefitinib and erlotinib, which are used to treat non-small cell lung cancer, and whose target is the epidermal growth factor receptor (EGFR). As such, screening methods for drugs having simultaneous inhibitory potential on the wild type and mutant targets are urgently needed. From the view point of an epidemiology study, the use of drugs that simultaneously inhibit the wild type and drug-resistant type targets can significantly reduce the probability of recurrence of mutation of mutant type targets. Additionally, from the view point of using clinical drugs, the use of drugs that have inhibitory potential against both the wild type and mutant type targets can avoid complex procedures associated with confirming the pathogenic strains, so as to significantly reduce the occurrence of delays treatment. Moreover, the use of drugs having inhibitory potential against both the wild type and mutant type targets can also reduce the occurrence of sides effects such as consensus interactions arising as a result of using lots of different drugs, or reduce the occurrence of inhibition against unexpected targets. The screening method of the present invention can screen out drugs that are effective against both wild type virus strains and drug-resistant virus strains, and is thus highly beneficial for drug development.

According to an embodiment, the target of drug screening method is NA. NA is a tetramer of identical subunits. In the influenza virus, NA is anchored on the surface of the viral envelope. In the process of the virus leaving the host cell, NA catalyzes the cleavage of terminal sialic acid residues to facilitate the release of progeny virions from infected cells. NA is thus a drug target for discovery of anti-influenza agents. However, mutant virus strains have been reported which confers drug resistance to current anti-influenza drugs. For instance, the H275Y, I223R and/or H275Y mutation significantly reduces the efficacy of the anti-influenza drugs such as GS4071 and zanamivir.

Candidates having inhibitory potential on NA are screened out by the drug screening method of the present invention. Candidates having inhibitory effects on NA are screened out by the drug screening method of the present invention. More specifically, drugs that can inhibit wild type and mutant type NA are screened out by the drug screening method of the present invention. The occurrence of certain NA mutations causes drug-resistance. Therefore, the drug screening method of the present invention is used for screening out drugs that have simultaneous efficacy on both wild type virus strains and drug-resistant virus strains. According to an embodiment, the drug screening method of the present invention can screen out drugs that have efficacy on both wild type virus strains and multiple drug-resistant virus strains, and is thus beneficial for drug discovery as well as having great clinical applicability.

According to the present invention, the drug screening method of the present invention can screen out drugs for the treatment of NA related diseases or disorders (such as influenza). According to an embodiment, the aforementioned drug screening method is used for screening out drugs for the treatment of diseases or disorders associated with wild type NA and/or mutant type NA. In one embodiment, the aforementioned drug screening method is used for screening out drugs for the treatment of diseases or disorders associated with wild type NA and/or drug-resistant NA. In one embodiment, the drug screening method of the present invention is used for screening out drugs for the treatment of diseases or disorders associated with wild type NA and/or multiple drug-resistant NA.

The drug screening method of the present invention is used for screening out drugs that can simultaneously inhibit wild type virus strains and mutant type virus strains. Thus, the drug screening method of the present invention is beneficial for drug development and has great clinical applicability. According to an embodiment, drugs that are effective in inhibiting both wild type virus strains and drug-resistant virus strains are screened out by the drug screening method of the present invention. In one embodiment, drugs that have inhibitory effects on both wild type virus strains and multiple drug-resistant virus strains are screened out by the drug screening method of the present invention.

According to an embodiment, the drugs that are simultaneously effective in the treatment of influenza caused by wild type influenza virus strains and mutant type influenza virus strains are screened out by the drug screening method of the present invention. In one embodiment, the drugs that are effective in the treatment of influenza caused by wild type influenza virus strains and drug-resistant influenza virus strains are screened out by the drug screening method of the present invention. In one embodiment, drugs effective in the treatment of influenza caused by wild type influenza virus strains and multiple drug-resistant influenza virus strains are screened out by the drug screening method of the present invention.

A compound of formula (I) is used for the preparing an inhibitor of NA:

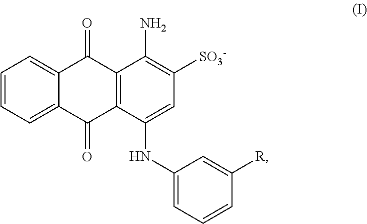

wherein R is one selected from the group consisting of:

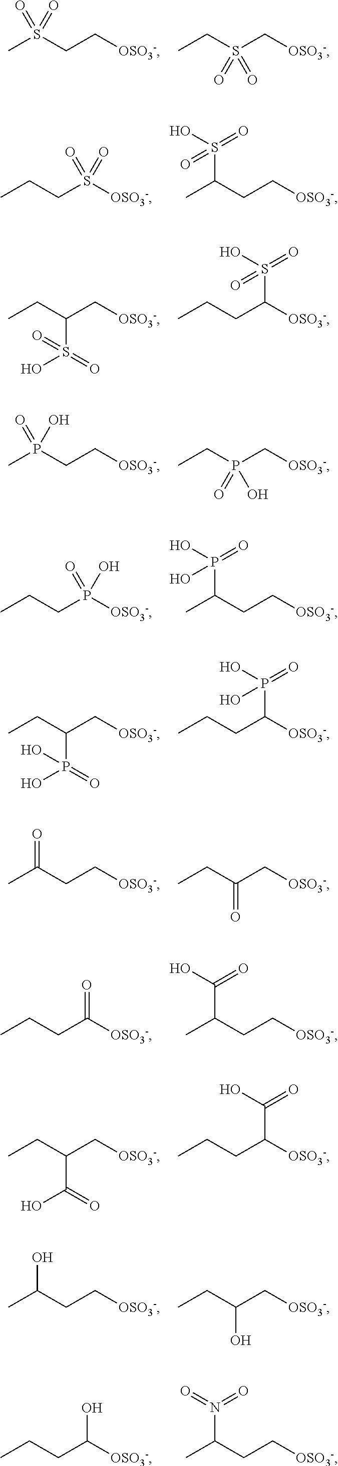

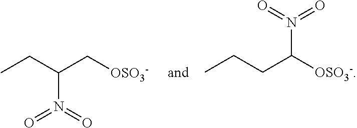

The above compounds of formula (I) for the preparation of NA inhibitors also comprise their salts, derivatives and analogues.

NA comprises wild type NA and mutant type NA. In one embodiment, the compound of formula (I) has inhibitory activity against wild type and mutant type NA.

In one embodiment, R in the compound of formula (I) is

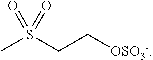

According to an embodiment, the compound of formula (I) has inhibitory effects against wild type virus strains and mutant type virus strains. In one embodiment, the compound of formula (I) has inhibitory effects against wild type virus strains and drug-resistant virus strains. In one embodiment, the compound of formula (I) has inhibitory effects against wild type virus strains and multiple drug-resistant virus strains.

The compound of formula (I) is used for the preparation of drugs for the treatment of influenza

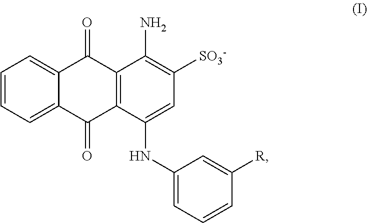

wherein R is one selected from the group consisting of:

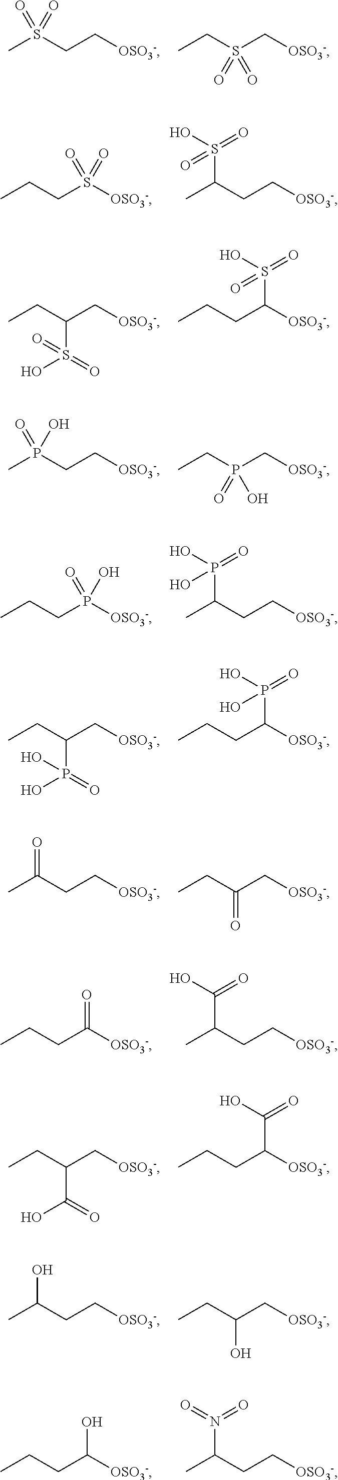

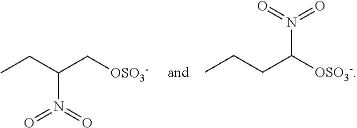

The aforementioned compound of formula (I) used for the preparation of drugs for the treatment of influenza comprises its salts, derivatives and analogues.

The compound of formula (I) has inhibitory activity on NA. The NA comprises wild type NA and mutant type NA. In one embodiment, the compound of formula (I) has inhibitory activity on wild type and mutant type NA. In one embodiment, R in the compound of formula (I) is as follows:

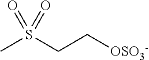

According to an embodiment, the compound of formula (I) has inhibitory effects against wild type virus strains and mutant type virus strains. In one embodiment, the compound of formula (I) has inhibitory effects against wild type virus strains and drug-resistant virus strains. In one embodiment, the compound of formula (I) has inhibitory effects against wild type virus strains and multiple drug-resistant virus strains.

In one embodiment, the compound of formula (I) is used for the preparation of drugs for treating influenza caused by influenza A virus. The influenza A virus comprises human influenza A virus, bovine influenza A virus, equine influenza A virus, swine influenza A virus and avian influenza A virus. In one embodiment, the influenza A virus comprises, but are not limited to, H1N1, H3N2 and H5N1 virus strains.

According to one embodiment, the compound of formula (I) has efficacy for simultaneously treating influenza caused by wild type influenza virus strains and mutant type influenza virus strains. In one embodiment, the compound of formula (I) has efficacy for simultaneously treating influenza caused by wild type influenza virus strains and drug-resistant type influenza virus strains. In one embodiment, the compound of formula (I) has efficacy for simultaneously treating influenza caused by wild type influenza virus strains and multiple drug-resistant type influenza virus strains.

The present invention provides a method of inhibiting NA. The method includes administering to a subject a therapeutically effective amount of a compound of formula (I)

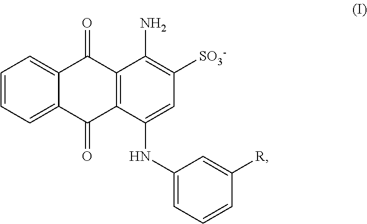

wherein R is one selected from the group consisting of:

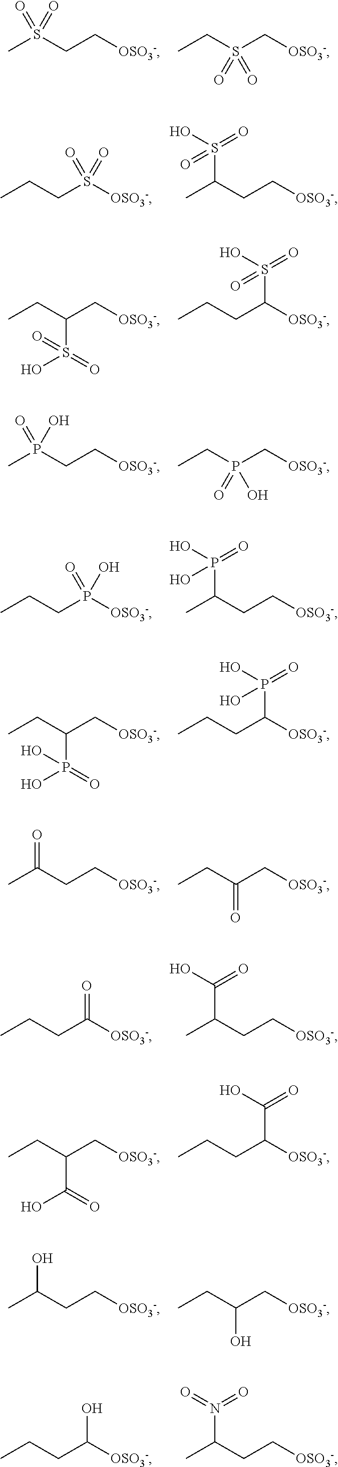

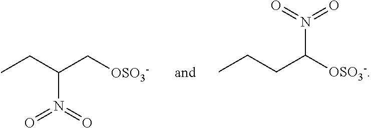

The above-mentioned compound of formula (I) used for inhibiting NA comprises its salts, derivatives and analogues.

NA comprises wild type NA and mutant type NA. In one embodiment, the compound of formula (I) has inhibitory activity on wild type and mutant type NA. In one embodiment, the compound of formula (I) has inhibitory effects on wild type NA and multiple drug-resistant NA. In one embodiment, the R in the compound of formula (I) is as follows:

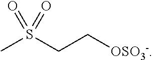

According to an embodiment, the compound of formula (I) has inhibitory effects on wild type virus strains and mutant type virus strains. In one embodiment, the compound of formula (I) has inhibitory effects on wild type virus strains and drug-resistant type virus strains. In one embodiment, the compound of formula (I) has inhibitory effects on wild type virus strains and multiple drug-resistant type virus strains.

The present invention provides a method of treating influenza. The method includes administering to a subject a therapeutically effective amount of a compound of formula (I):

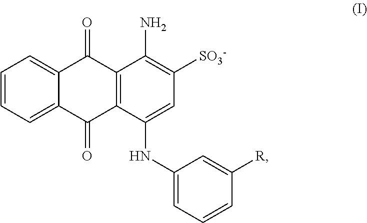

wherein R is one selected from the group consisting of:

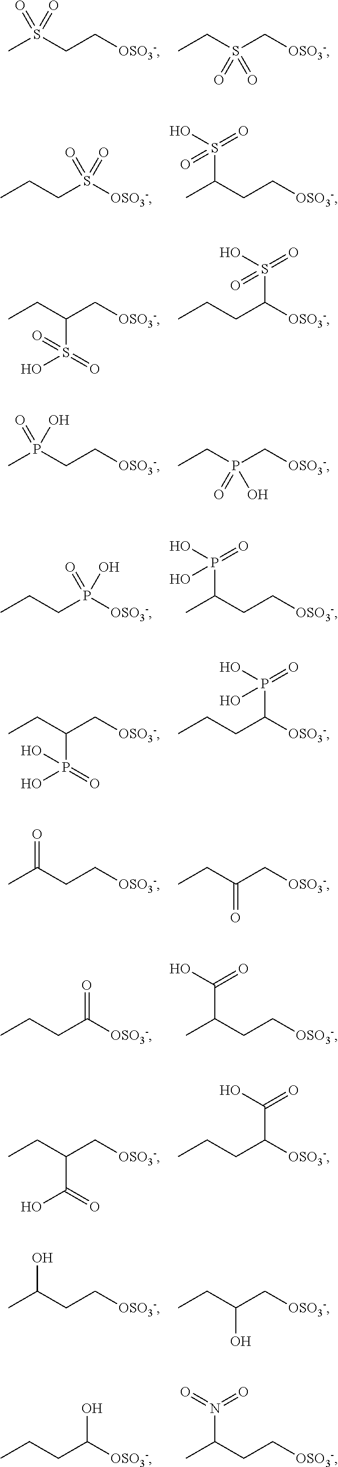

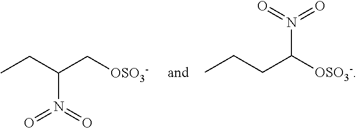

The compound of formula (I) above for treating influenza comprises its salts, derivatives and analogues.

The compound of formula (I) has inhibitory activity on NA. NA comprises wild type NA and mutant type NA. In one embodiment, the compound of formula (I) has inhibitory activity on wild type and mutant type NA. Preferably, R in the compound of formula (I) is

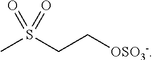

According to an embodiment, the compound of formula (I) has inhibitory effects on wild type virus strains and mutant type virus strains. In one embodiment, the compound of formula (I) has inhibitory effects on wild type virus strains and drug-resistant virus strains. In one embodiment, the compound of formula (I) has inhibitory effects on wild type virus strains and multiple drug-resistant virus strains.

According to an embodiment, the compound of formula (I) is used for treating influenza caused by influenza A virus. The influenza A virus comprises human influenza A virus, bovine influenza A virus, equine influenza A virus, swine influenza A virus and avian influenza A virus. In one embodiment, the influenza A virus comprises, but are not limited to, H1N1, H3N2 and H5N1 virus strains.

In one embodiment, the compound of formula (I) has efficacy for treating influenza caused by the wild type influenza virus strain and mutant type influenza virus strains. In one embodiment, compound of formula (I) has efficacy for treating influenza caused by the wild type influenza virus strain and drug-resistant influenza virus strains. In one embodiment, compound of formula (I) has efficacy for treating influenza caused by the wild type influenza virus strain and multiple drug-resistant influenza virus strains.

The present invention provides a pharmaceutical composition having the compound of formula (I) as mentioned above. In one embodiment, R in the compound of formula (I) is as follows:

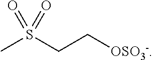

The compound of formula (I) comprises its salts, its derivatives and its analogues. In the pharmaceutical composition, the amount of the compound of formula (I), its salts, derivatives or analogues is completely within the medically-determined range, and is sufficient to demonstrate a positive improvement in symptoms, diseases or disorders (this amount can also be known as a therapeutically effective amount). Further, the amount can also be modified to prevent severe side effects. In other words, the effective amount of the compound of formula (I), its salts, derivatives or analogues in the pharmaceutical composition varies with the desired aims of treatment, the health condition and age of subjects to be treated, the severity of original diseases, duration of treatment, nature of treatment, and specific drugs to be used.

In an embodiment, the above-mentioned pharmaceutical composition has inhibitory activity on wild type NA and mutant type NA. In one embodiment, the pharmaceutical composition has inhibitory activity on wild type NA and drug-resistant NA. In one embodiment, the pharmaceutical composition has inhibitory activity on wild type NA and multiple drug-resistant NA. In one embodiment, the pharmaceutical composition has inhibitory effects on wild type influenza virus strains and mutant type influenza virus strains. In one embodiment, the pharmaceutical composition has inhibitory effects on wild type influenza virus strains and drug-resistant influenza virus strains. In one embodiment, the pharmaceutical composition has inhibitory effects on wild type influenza virus strains and multiple drug-resistant influenza virus strains.

According to an embodiment, the pharmaceutical composition is used for treating influenza, particularly influenza caused by influenza A virus. The influenza A virus comprises human influenza A virus, bovine influenza A virus, equine influenza A virus, swine influenza A virus and avian influenza A virus. In one embodiment, the influenza A virus comprises, but are not limited to, H1N1, H3N2 and H5N1 virus strains.

According to an embodiment, the pharmaceutical composition has inhibitory effects on wild type virus strains and mutant type influenza virus strains. In one embodiment, the pharmaceutical composition has treatment efficacy on influenza caused by on wild type influenza virus strains and drug-resistant influenza virus strains. In one embodiment, the pharmaceutical composition has treatment efficacy on influenza caused by wild type influenza virus strains and multiple drug-resistant influenza virus strains.

### EMBODIMENTS

The methods implemented by the present invention are illustrated by the following Embodiments, but these methods are not intended to limit the scope of the present invention. The other advantages and effects of the present invention can also be understood by persons skilled in the art according to the disclosure of the present invention. The present invention can also be implemented or used together with the other different embodiments.

Unless otherwise stated in the specification, the singular form expressions “a” and “the” in the specification and the claims include one and plural individuals. Unless otherwise stated in the specification, the term “or” in the specification includes the definition of “and/or”.

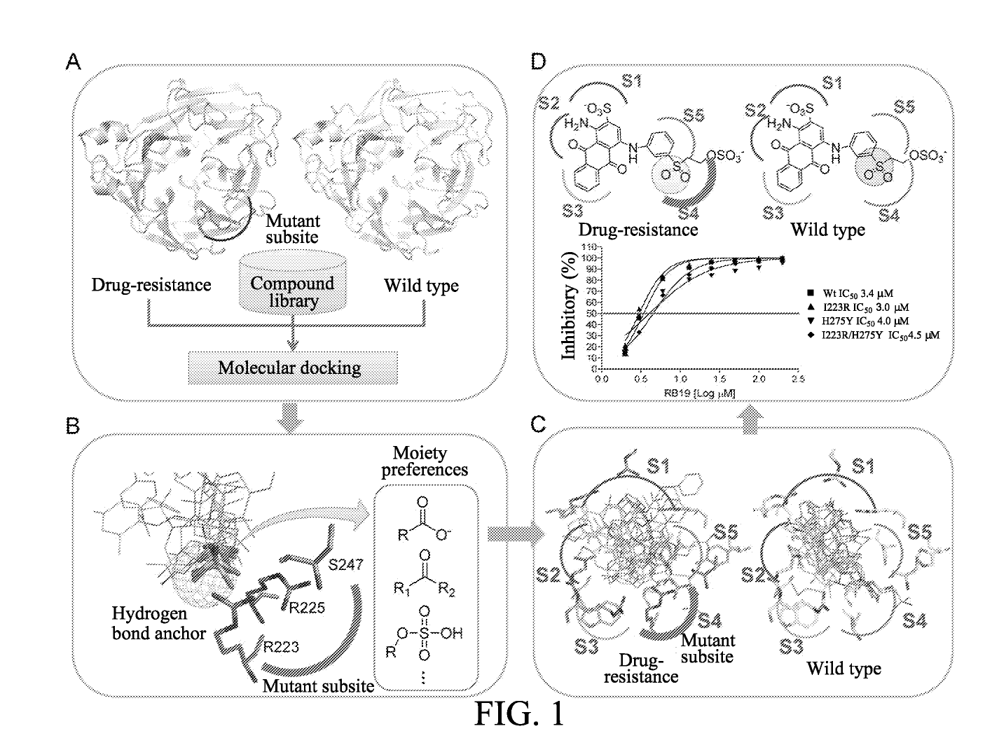

FIG. 1 shows a framework of the parallel screening strategy for identifying inhibitors of wild type NA (neuraminidase) and mutant type NA (exemplified in the Embodiments as drug-resistant; for example multiple drug-resistant neuraminidase) Briefly, 257,275 compounds selected from public compound databases were docked to binding sites of wild type and multiple drug-resistant NA using the in-house docking tool, GEMDOCK (FIG. 1A). GEMDOCK was used successfully as a molecule docking tool, and was also successfully applied to identify novel inhibitors and binding sites for several targets in the art. After the docking procedure, characterization of binding sites of the wild type target and mutant type target was carried out. For instance, after this procedure, the binding sites of the docked compounds were characterized using the site-moiety map (SiMMap), wherein the characterization includes characterization of the mutant subsite. The site-moiety map demonstrated the relationship between moiety preferences and physico-chemical properties of the binding sites through anchors (FIG. 1B).

In the present invention, the NA binding site was divided into 5 subsites, i.e. S1 (R118, R293, and R368 belonging to the N1 series), S2 (E119, D151, W179, and E228), S3 (R152, W179, and I223), S4 (I223, R225, and S247), and S5 (S247 and E277). Characteristics of the subsites of wild type NA were described previously, and by combining the characteristics of the mutant subsite, the compounds that simultaneously matched characteristics of wild type and multiple drug-resistant subsites were selected (FIG. 1C). Then, the bio-assays of wild type and multiple drug-resistant NA enzyme activity in the presence or absence of test compounds were performed (FIG. 1D).

### Preparation of Neuraminidase Structures and Screening Library

For the parallel screening strategy, the open-form H5N1 NA structure (PDB code: 2HTY) was downloaded from the Protein Data Bank. This structure that was not in complex with ligands was selected because ligand-bound structures may have induced fit, and may restrict the diversity of identified inhibitors. To define the binding site of NA, the zanamivir-bound structure (PDB code: 2HU4) was aligned to this unbound structure (i.e. 2HTY) using a structural alignment tool. The binding site was defined as residues within a 10 Å radius sphere centered around the zanamivir.

The NA structure with I223R and H275Y dual-point mutations was derived using a homology-modeling server. The protein sequence submitted for the server was from the strain NIBRG14 (H5N1) with the dual-point mutation, which was used to provide multiple drug-resistant NA for the bio-assay. The unbound NA structure (i.e., 2HTY) was selected as the structure template. The binding site of the mutant structure was generated using the procedure described above.

Compound libraries used for virtual screening include databases from the National Cancer Institute (NCI) and Sigma-Aldrich (St. Louis, Mo.). Small molecular compounds having molecular weights between 200 and 650 daltons were selected for virtual screening as described below (that is, performing screening using the molecular docking tool). According to statistics, about 80% of drugs have molecular weights of between 200 and 650 daltons, thus indicating that compounds within this range may have more preferable drug kinetics (absorption, distribution, metabolism, excretion), and are thus more likely to become drugs. The number of selected compounds was 257,275.

### Virtual Screening

Each compound was docked into the binding site of NA using the docking tool, GEMDOCK. This tool rapidly measured intermolecular potential energies between binding sites and compounds using a scoring function that is based on piecewise linear potential. The scoring function of GEMDOCK contained electrostatic, steric, and hydrogen-bonding potentials, and the intermolecular potential energy for protein-compound complexes was calculated as the sum of the three potentials. After docking, the candidates were ranked based on their intermolecular potential energies. Each compound was docked into the binding site of the wild type NA. The top 12,800 compounds (approximately 5% of compounds) were docked into the binding sites of multiple drug-resistant NA, in order to obtain energy potential rankings.

### Characterization—Site-Moiety Map and Interaction Profile Analyses

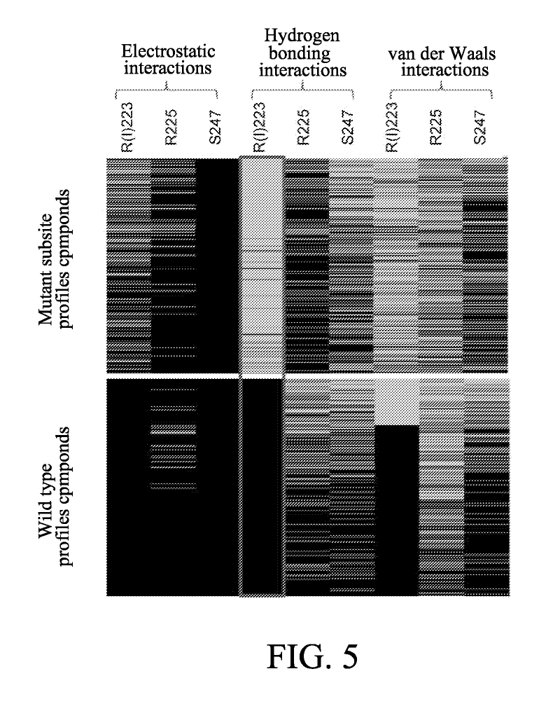

The compounds were docked to the binding sites of wild type NA as described above, and 600 compounds (a second group of compounds) which interacted with the S4 subsites of mutant NA (mutant subsites) were selected from the compounds with higher rankings (in a first group of compounds) by means of the docking tool. Further analysis was performed to analyze the moiety and interaction preferences of the subsite. These compounds and binding sites of the wild type and multiple drug-resistant NA were used to establish site-moiety maps. In theory, at least 500 compounds were required to establish a site-moiety map. First, protein-compound interaction profiles were used to present interactions between compounds and protein residues. The three profile types used included electrostatic (E), hydrogen-bonding (H), and van der Waals (V) interaction profiles. For each profile, the interactions were represented by a matrix with size P×C, wherein P and C were the compound number and the interacting residue number of the protein, respectively. Interactions were detected using the piecewise linear potential function of GEMDOCK. Profiles were then visualized by iGEMDOCK (which is graphical interface version of GEMDOCK (tools such as EXCEL can also be used to visualize the profiles). The entry of the E and H profiles was set to 1 if there were electrostatic or hydrogen-bonding interactions between the compound and the residue (gray regions in FIG. 5); otherwise the entry was set to 0 (black regions in FIG. 5). For the V profile, entry was set to 1 if its V interaction energy was less than −4 kcal/mol.

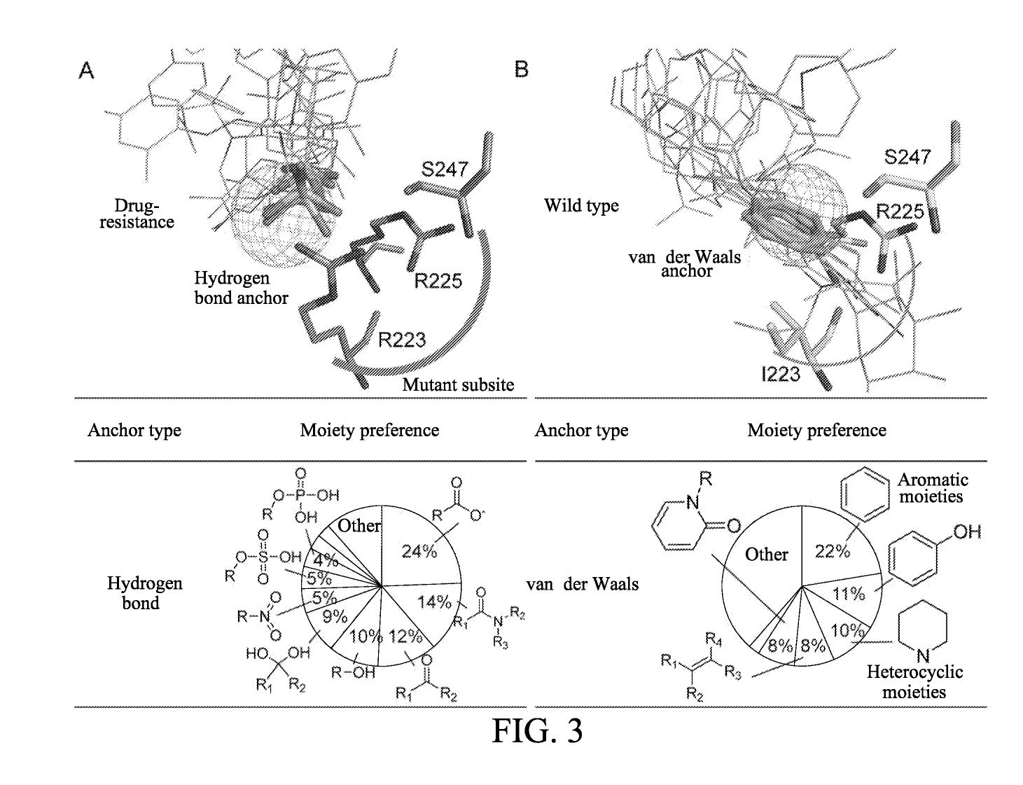

Consensus interaction sites of the profiles were recognized using Z-scores as anchors, which often played important roles in biological functions. For each profile, the Z-score value (Zᵢ) of the protein residue i was obtained according to the equation:

$${Z}_{i} = \frac{{f}_{i} - μ}{σ} ,$$

wherein fᵢ is the observed interaction frequency between the compounds and the residue i, and μ and σ are the mean and standard deviation of interaction frequency acquired from 1,000 randomly shuffled profiles. Interactions between compounds and the residue i with a Z-score of ≧1.645, which was commonly used as a statistical threshold (95% confidence level), were regarded as consensus interactions. In this way, spatially neighboring residues with consensus interactions and their interacting moieties consisted of an anchor. In one embodiment of the present invention, the anchor located at the S4 subsite was used to characterize the subsites of wild type and multiple drug-resistant NA (FIG. 3).

### Characterization of Mutant Subsite

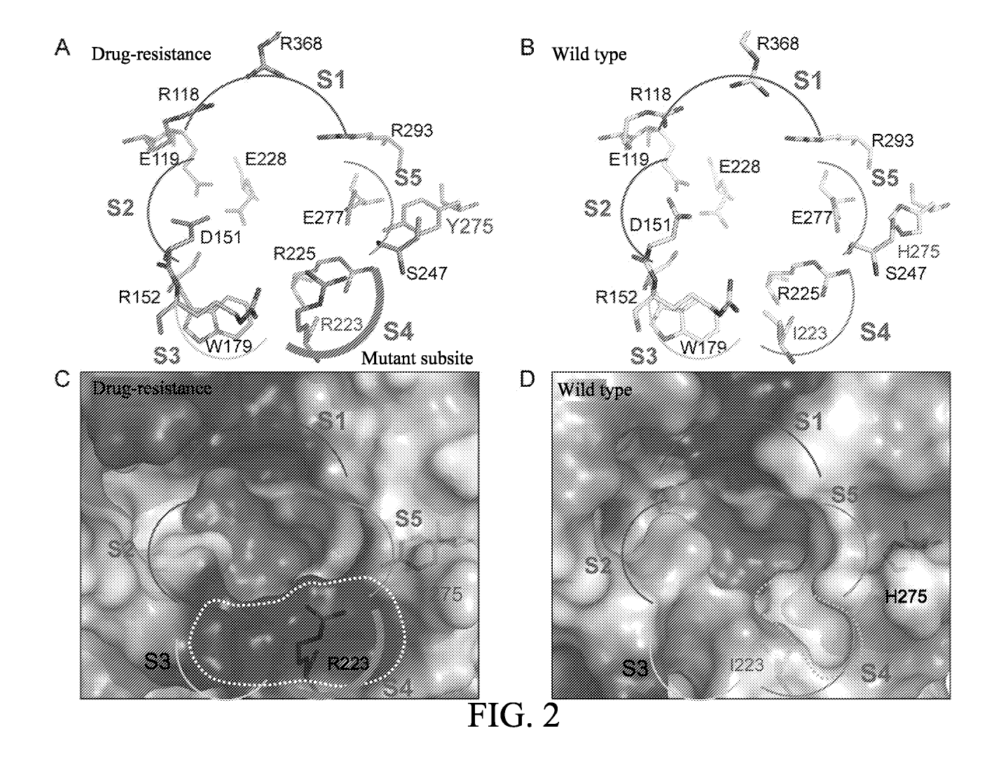

Structural comparisons of wild type and multiple drug-resistant NAs showed striking differences in volume and polarity of the S4 subsites, which were mainly caused by the I223R mutation (FIG. 2). Due to the long side-chain of arginine, the volume of the S4 subsite was reduced. Furthermore, within the mutant subsite, the residue R223 and its neighboring arginines (R152 and R225) formed a positively-charged region (FIG. 2C), whereas the wild type S4 subsite was hydrophobic (FIG. 2D). The reduced volume and changed characteristic of the S4 subsite indicated that inhibitors containing long lipophilic side chains (e.g., oseltamivir) or aromatic rings (e.g., carbocyclic analogue 53) were inappropriate as inhibitors of multiple drug-resistant NA.

The site-moiety map analyses revealed that a hydrogen-bonding anchor consisting of the three residues R223, R225 and S247 was located at the mutant S4 subsite (FIG. 3). The anchor preferred polar moieties such as carboxylic acid, amide, ketone, and sulfuric acid. In contrast, the anchor-type located at the wild type subsite was van der Waals interaction, and this anchor preferred ring moieties such as aromatic, phenyl, heterocyclic, and alkene. The difference in moiety preference may be caused by reduced volume and positive charge of the region. Based on these observations, inhibitors had large polar moieties (for example sulfuric acid derivatives and phosphoric acid derivatives) in the S4 subsite, and this may imply a useful design for maintaining activity against both wild type and multiple drug-resistant NAs. Such moieties were able to provide van der Waals contacts with the WT subsite. Moreover, when the dual mutation arises, these moieties may yield hydrogen-bonding interactions with the polar environment.

### Interaction Preference of the Mutant Subsite

Understanding interaction preferences of protein subsites facilitates the discovery of inhibitors and the study of ligand binding mechanisms. To understand the interaction preferences of the mutant subsites, the interaction profiles of the top 600 compounds (the second group of the compounds) for wild type and multiple drug-resistant NA were analyzed using iGEMDOCK (which is GEMDOCK graphical interface version) (FIG. 5). This tool is a graphical environment used to enhance GEMDOCK for protein-compound interaction visualization and post-screening analysis. Such analysis revealed that many atoms of the top compounds formed hydrogen bonds with the mutant subsite, and whereas the wild type subsites had relatively few hydrogen-bonding interactions (Table 1). The short side-chain of I223 made a large cavity, resulting in more favorable van der Waals interactions with bulky moieties (sometimes known as groups) than the mutant subsite. The strong activity of GS4071 was obtained by optimizing van der Waals interactions with the wild type subsite.

These interaction profiles showed that the major differences between the interaction preferences of the two kinds of subsites were dependent on hydrogen-bonding interactions (FIG. 5 and Table 1). For example, there were 95% and 0% of compounds respectively having hydrogen bonds with the multiple drug-resistant subsite residue R223 and the wild type subsite residue I223 (Table 1). The high preference to form hydrogen bonds may account for RB19's (described in later section) potent activity against the dual-mutant NA. In addition, it was observed that some compounds had electrostatic interactions with R152 and R223 of the mutant subsite, which suggested that inhibitors with negatively-charged moieties (e.g., sulfuric acid monoester, phosphoric acid, and carboxylic acid) may enhance potency due to salt-bridge formation.

TABLE 1
Interaction preferences of wild type subsite and mutant subsite

| | | | | Hydrogen-bonding | | | | | |
|---|---|---|---|---|---|---|---|---|---|
| Type of Interaction | Electrostatic interactions | | | interactions | | | van der Waals interactions | | |
| Residue | R(I)223 | R225 | S247 | R(I)223 | R225 | S247 | R(I)223 | R225 | S247 |
| % Interaction (Mutant type) | 31% | 9% | 0% | 95% | 18% | 46% | 75% | 57% | 31% |
| % Interaction (Wild type) | 0% | 4% | 0% | 0% | 27% | 28% | 21% | 57% | 25% |

### Identification of Anti-Resistance Inhibitors

Compounds that simultaneously match characteristics of the binding sites of wild type and multiple drug resistant NA were selected based on interactions and shape complementarity. Subsequently, these compounds were evaluated for their anti-NA activity. The S1, S2, S3 and S5 subsite characteristics were known directly from the research of Stoll et al. (Stoll V, Stewart K D, Maring C J, Muchmore S, Giranda V, et al. (2003) Influenza neuraminidase inhibitors: Structure-based design of a novel inhibitor series. Biochemistry 42: 718-727). The S1 subsite was a positively charged environment. Further, the compound and the subsite generated electrostatic interaction. The S2 subsite environment was negatively charged. If a compound and the S2 subsite environment generated electrostatic interaction, then the compound will be considered as matching the subsite. For the two aforementioned subsites, when the compound and the subsite formed a hydrogen-bonding interaction, this was then considered as characteristic matching. The S3 subsite was a hydrophobic and polar region, and it can also have hydrogen-bonding interactions or van der Waals interactions. The S5 subsite was a hydrophobic region, and the compounds that formed van der Waals interactions with this subsite had the same characteristics as this subsite.

The parallel matching scores were used to identify compounds that simultaneously had inhibitory activity against wild type and multiple drug-resistant NA. For compound c, its parallel matching score was calculated as

$$PMS ( c ) = {\sum }_{s = 1}^{S} {WS}_{s} ( c ) + {\sum }_{s = 1}^{S} {MS}_{s} ( c ) + ( - 0.001 ) \frac{WE ( c )}{{M}^{0.5}} + ( - 0.001 ) \frac{ME ( c )}{{M}^{0.5}} ,$$

wherein WSₛ(c) and MSₛ(c) were the matching score of compound c in the subsite s of wild type and multiple drug-resistant NA, respectively, S was the number of subsites number, WE(c) and ME(c) represented the intermolecular potential energies between compound C and wild type NA and the intermolecular potential energies between compound C and multiple drug-resistant NA, respectively, and M represented the number of atoms of compound C. Here, WSₛ(c) or MSₛ(c) was set to 1 if the compound c matched the characteristic of the subsite s, otherwise, the score was set to 0. M^{0.5} may reduce that compounds with larger molecular weights were preferentially selected due to the score function of energy basis. The compounds were then ranked based on the parallel matching scores. In order to find compounds that were not affected by dual mutation, compounds were filtered if they did not match the characteristic of the mutant S4 subsite. Then, the selected compounds were tested using the bio-assay.

As defined by Stoll et al., the binding sites were divided into 5 subsites (S1-S5) (FIG. 2). The S1 subsite (R118, R293, and R368) was a positively-charged region, and many inhibitors such as zanamivir and oseltamivir carboxylate (GS4071) interacted with this subsite through carboxylic acid moieties. The S2 subsite was composed of residues E119, D151, W179 and E228, and was a negatively-charged environment that interacted with the guanidine of zanamivir through hydrogen bonds. The three residues R152, W179, and I223 of the S3 site possessed long side-chains. The crystal structures of protein-compound complexes (PDB codes: 3B7E, 2HU4, and 1MWE) indicated that the acetamido moieties of sialic acid, zanamivir and GS4071 formed hydrogen bonds with R152 of the subsite. The S4 (I223, R225 and S247) and S5 (S247 and E277) subsites of wild type NA were hydrophobic. van der Waals interactions between the two subsites and GS4071 were essential for binding of this inhibitor. It should be noted that the S4 subsite environment changed from hydrophobic to polar environment when the dual mutation arose. Since the subsites played important roles in NA inhibitor binding, the compounds that simultaneously interacted with the subsites of wild type and multiple drug-resistant NA were considered as potential anti-resistance inhibitors.

Parallel matching scores were used to confirm that the compound of formula (I) had inhibitory potential against both wild type and multiple drug-resistant type NA.

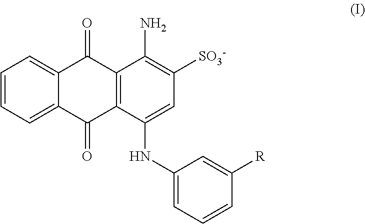

In the formula (I), R was one selected from the group consisting of:

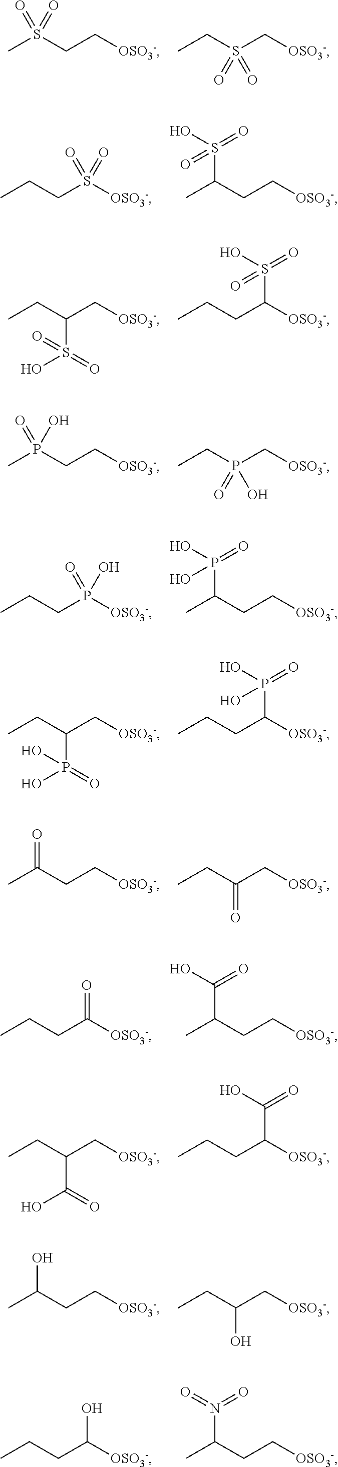

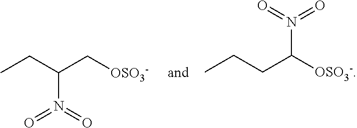

According to an embodiment, in order to improve its efficacy, the structure of compound of formula (I) can be modified. In one embodiment, modification was made on the groups that mediated interaction between the residues of subsites of binding sites and the compound of formula (I), in order to improve inhibitory efficacy. In one embodiment, modification was made on compound (I), particularly on the groups of residues of the binding sites' subsites thus allowing more hydrogen bonding formation and increasing the efficacy of compound (I). In one embodiment, in order to improve the efficacy of the compound of formula (I), the structure of the compound of formula (I) was modified with various groups such as guanidine group, amino group, their analogues and/or derivatives and the like.

### Compound Selection for Bioassay

Compounds that simultaneously matched the subsite characteristics of wild type and multiple drug-resistant NA were selected because these compounds maintained their potency despite mutations of the enzyme. A compound was considered as matching a subsite if its interacting moiety was physically and chemically complementary to the subsite, and if it formed appropriate interactions with the subsite. For example, based on the site-moiety map analyses, the S4 subsites of multiple drug-resistant and wild type NA preferred hydrogen-bonding and van der Waals interactions, respectively. Once the compounds that matched all subsite characteristics of wild type and multiple drug-resistant NA were selected, bio-assay was performed on these selected compounds.

In the compound of formula (I), R was

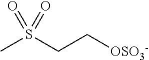

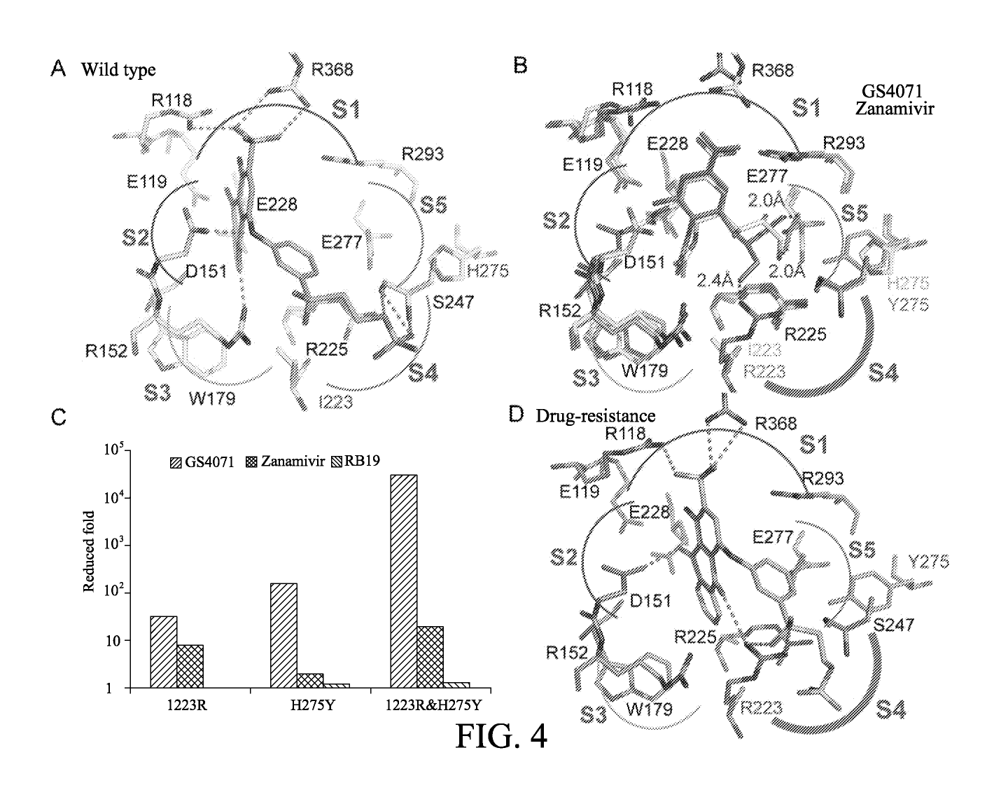

(remazol brilliant blue R, RB19, RB19, an anthraquinone dye). The sulfone moiety of RB19 formed hydrogen-bonding interactions with the mutant S4 subsite, and RB19 was thus considered as matching the characteristic of the S4 subsite. RB19 was an anti-resistance inhibitor that was active against both wild type and multiple drug-resistance NA. This compound inhibited the NA of influenza NIBRG14 (H5N1) with an IC₅₀ of 5.7 μM (Table 2), and its docking conformation revealed similar interactions with the 5 subsites as those of zanamivir and GS4071 (FIGS. 4A and 4B). The sulfonate moiety of RB19, which had similar physico-chemical properties as the carboxylic acid moieties of zanamivir and GS4071, formed electrostatic interactions with R118 and R368 in the S1 subsite. The electrostatic interactions between negatively-charged moieties and positively-charged residues were consistent with NA complexed with known ligands including sialic acid, zanamivir, and GS4071 (PDB codes: 3B7E, 2HU4, and 1MWE). In the S2 subsite, the dimethylamine of RB19 yielded a hydrogen-bonding interaction with D151, which played a role similar to that of the guanidine group of zanamivir. According to an embodiment, the structure of RB19 can be modified in order to improve its inhibitory efficacy. In one embodiment, the modification made to the structure of RB19 improved its binding efficacy with binding site, and thus improving its efficacy. In one embodiment, RB19 was modified with groups which enhanced the interactions with residues of the subsites in the binding sites, so as to improve its efficacy. In one embodiment, RB19 was modified with the groups which formed more hydrogen bonds with residues of the subsites in the binding sites, thus increasing efficacy of RB19. In one embodiment, structural modifications of RB19 (for example, addition of guanidine group, amino group, their analogues and/or derivatives and the like) can increase efficacy of RB19. To further demonstrate this, the addition of guanidine moiety to RB19 provided six hydrogen-bonding interactions between the residues E119, D151, W179 and E228 in the S2 subsite, thus increasing its efficacy.

Within the S3 subsite, the ketone in the tetrahydroanthracene moiety of RB19 occupied a similar position to the acetamido moiety of zanamivir and GS4071. This ketone moiety had interaction with R152 through hydrogen-bonding (FIG. 4A). Similarly, the acetamido moieties of zanamivir and GS4071 formed hydrogen bonds with R152. In addition, tetrahydroanthracene made van der Waals contacts with the long side-chains of residues E117, D151, R152, W179 and E228 of the S2 and S3 subsites. This moiety, which was different to the acetamido group of GS4071 and zanamivir, may be advantageous to NA inhibitors. Similar to the 3-pentyloxy group of GS4071, the sulfone moiety on the aromatic ring of RB19 also formed van der Waals contacts with residues in the S4 subsite (FIGS. 4A and 4B). In addition, the sulfuric acid monoester of RB19 formed a hydrogen-bonding interaction with S247 of the S5 subsite.

The multiple drug-resistant inhibitory activity of RB19 was further examined. In addition, NAs with the respective single mutation (that is, neuraminidase^{I223R} and neuraminidase^{H275}) were used to evaluate the efficacy of RB19. An insect cell protein expression technology was employed to express these NAs for studying their sensitivity to RB19. First, GS471 and zanamivir were used for testing these mutant NAs. The experimental results showed that the mutant neuraminidase^{I223R and H275}, neuraminidase^{I223R} and neuraminidase^{H275} had 8- to >20.000-fold decreased susceptibility to GS4071, and had up to 2- to 36-fold decreased susceptibility to zanamivir. In comparison, the IC₅₀ of RB19 for neuraminidase^{wt}, neuraminidase^{I223R and H275}, neuraminidase^{I223R} and neuraminidase^{H275} activity were 3.4, 4.5, 3.0, and 4.0 μM, respectively (Table 2). The fold change shown in Table 2 also represented a reduced susceptibility (activity) ratio (FIG. 4C).

The docking conformation of RB19 revealed that two hydrogen-bonding interactions were yielded between the sulfone moiety and R223 of the mutant S4 subsite (FIG. 4D), which may account for the similar inhibition of multiple drug-resistant neuraminidase by RB19. The sulfone moiety was able to maintain its interactions with the S4 subsite when the environment changed from a large hydrophobic subsite to a small polar subsite. In contrast, the mutant S4 subsite may not accommodate the 3-pentyloxy group of GS4071 or the glycerol side chain of zanamivir (FIG. 4B). As such, these two inhibitors had reduced potency. For GS4071, two clashes were observed between R223 and the 3-pentyloxy group (2.4 Å), and between E277 and the 3-pentyloxy group (2.0 Å). The glycerol moiety of zanamivir was relatively distant from R223, and the hydrogen bonds between the glycerol moiety and E277 were preserved, leading to a reduction in inhibitory activity. Because of the I223R mutation, the ketone moiety of tetrahydroanthracene switched its hydrogen-bond partner from R152 of the S3 subsite to R223 of the S4 subsite.

The compound RB19 comprised a rigid core scaffold, 1,4-diamino-9,10-dioxoanthracene-2-sulfonate, and a flexible side chain; 2-(3-methylphenyl)sulfonylethyl hydrogen sulfate, and both of these were good starting points for designing anti-resistance inhibitors. The core scaffold formed electrostatic, hydrogen-bonding, and van der Waals interactions with the S1, S2, and S3 subsites in both wild type and multiple drug-resistant NAs, respectively (FIGS. 4A and 4D). Because the residues R118, D151, and R368 of the S1, S2, and S3 subsites were highly conserved in all NA subtypes, and directly interacted with the substrate sialic acid, mutations on these sites may induce a loss of NA activity. This indicated that the subsites had a decreased probability of acquiring resistance, and that the core scaffold was promising for interacting with these conserved regions. Unlike the high conservation of the S1, S2, and S3 subsites, the S4 subsite had relatively low residue conservation, and acquired drug resistant mutations such as H275Y and I223R. The 2-(3-methylphenyl)sulfonylethyl hydrogen sulfate moiety had the potential to be applied in the design of anti-resistant drugs, because its flexible side chain can tolerate the volume change induced by mutations of S4 residues. The flexible side chain formed van der Waals contacts with the wild type S4 subsite. When mutations arose, it changed its orientation to yield hydrogen bonds with the S4 subsite of the multiple drug-resistant type NA. These interactions maintained the inhibitory activity of RB19, which was similar to that observed in wild type NA. These results revealed that RB19 and the two scaffolds were useful inhibitor of multiple drug-resistant NA, and they were therefore good starting points for the design of new multiple drug-resistant NA inhibitors.

### Drugs and Reagents

The selected compounds which were requested or purchased, were respectively dissolved in dimethyl sulfoxide (DMSO) and stored at −20° C. Oseltamivir carboxylate (GS4071) was synthesized by Dr. Kak-Shan Shia at the National Health Research Institutes of Taiwan. The fluorogenic substrate 2′-(4-methylumbelliferyl)-α-d-N-acetylneuraminic acid (MU-NANA) was obtained from Sigma-Aldrich.

### Viruses and Cells

The influenza strains A/WSN/33 (H1N1) and A/Udorn/72 (H3N2) were used. Two clinical isolates that were resistant to GS4071, A/TW/70058/09 (H1N1) and A/TW/70066/09 (H1N1), were utilized in the antiviral assay. Madin-Darby canine kidney (MDCK) cells were obtained from the American Type Culture Collection (Manassas, Va.) and were maintained in Dulbecco's Modified Eagle's Medium (DMEM) with 10% fetal bovine serum.

Generation of Oseltamivir- and Zanamivir-Resistant Neuraminidase with I1275Y and I223R Point Mutations

The N1 NA gene for strain NIBRG14 (H5N1) was used. The recombination plasmid pGEM-T easy containing the NA gene from strain NIBRG14 (H5N1) was used as a target for I223R site-directed PCR mutagenesis of NA. To construct the I223R and H275Y double mutation expression plasmid, recombinant plasmid pGEM-T easy that contains the single point mutation H275Y NA gene (H5N1) was used as the target for site-directed PCR mutagenesis of NA.

To generate I223R-specific mutations, two primer sets were designed as follows:

I223R-F;
(SEQ ID No. 1)
(5′-GAG TTG GAG GAA CAA CAG ACT GAG AAC TCA AGA
GTC TG-3′),
and
I223R-R;
(SED ID No. 2)
(5′-CAG CTC TTG AGT TCT CAG TCT GTT GTT CCT CCA
ACT C-3′).

Mutation sites were created by PCR using 0.4 mM dNTP, 0.3 μM primers, 2 ng template DNA, and an appropriate amount of AccuPrime™ Turbo Pfu DNA polymerase and buffer (Invitrogen, Carlabad, Calif.). The thermal cycling profile was as follows: 95° C. for 5 minutes, followed by 20 cycles of 94° C. for 30 seconds, 55° C. for 1 minute, and 68° C. for 8 minutes, and final extension at 68° C. for 7 minutes. After PCR amplification, DpnI was added to digest the plasmid template. Amplified DpnI-insensitive mutated plasmids were introduced into Escherichia coli for selection of the designed mutant sequences.

### Production of NA in Insect Cells

The NA expression constructs were co-transfected with linear BacPAK8 viral DNA into Sf9 insect cells. Culture media from infected cells was then collected and stored as virus stock for the production of NA for the enzyme assay. Recombinant baculoviruses—Bac-NA^{WT}, Bac-NA^{H275Y}, Bac-NA^{I223R}, and Bac-NA^{I223R and H275Y}—were generated to express wild-type, H275Y, H275Y, and I223R mutants of NA originating from influenza N1 NA (NIBRG14(H5N1)). Total cell lysates were treated with 2.5 mg/ml pronase for 1 hour at 22° C.

### Cell-Associated and Cellular Virus Yields

After infection of MDCK cells with MOI 0.001 A/WSN/33, various concentrations of compound were added to the cell media. After 48 hours, culture supernatants were taken, and infected cells were scraped off wells and collected by centrifugation at 16000 g for 5 minutes. To determine the extracellular and cell-associated virus titer after treatment with the inhibitors, supernatants and lysates were assayed for NA activity.

### Evaluation of Antiviral Activities of Neuraminidase Inhibitors

To measure the potential of identified inhibitors to inhibit cytopathic effects (CPE) in MDCK cells infected with influenza viruses, the CPE inhibition assay protocol described by Hung et al. (Huang H C, Tseng C P, Yang J M, Ju Y W, Tseng S N, et al. (2009) Aurintricarboxylic acid inhibits influenza virus neuraminidase. Antiviral Research 81: 123.131) was used. The concentration of compound required to reduce the CPE of the virus by 50% (IC₅₀) was determined. In these experiments, GS4071 was used as a positive control. Plaque assays were used to determine the effects of inhibitors on influenza virus replication, as described by Hung et al. The concentration of inhibitors required to reduce the number of plaques by 50% (EC₅₀) was then determined Cell toxicity of inhibitors was determined using an MTS assay. Optical density was measured by ELISA at OD 490 nm

### Neuraminidase Inhibition Assay

To inactivate viral infectivity, cell culture suspensions of virus-infected MDCK cells were inactivated with formaldehyde (0.02%) as described by Hung et al. Enzymatic activity of NA was measured using the fluorogenic substrate MU-NANA. To evaluate the inhibitory effects of identified compounds, inactivated virus supernatants were pre-incubated with the compounds for 30 minutes at 30° C. The assay was conducted in 96-well plates containing diluted virus supernatant (containing active influenza NA) and 100 μM fluorogenic substrate per well in an MES buffer (32.5 mM MES, 4 mM CaCl2 at pH 6.5). The enzyme reactions were then carried out for 1 hour at 37° C. and were terminated by adding a stop solution containing 25% ethanol and 0.1 M glycine (pH 10.7). Fluorescence intensity of the product 4-MU was measured using a Fluoroskan spectrofluorometer (Labsystems, Helsinki, Finland) with excitation and emission wavelengths of 330 and 445 nm, respectively. The IC₅₀ for NA activity was then determined using GS4071 as a positive control.

### Testing the Effect of RB19 Using Cell-Based Experiments

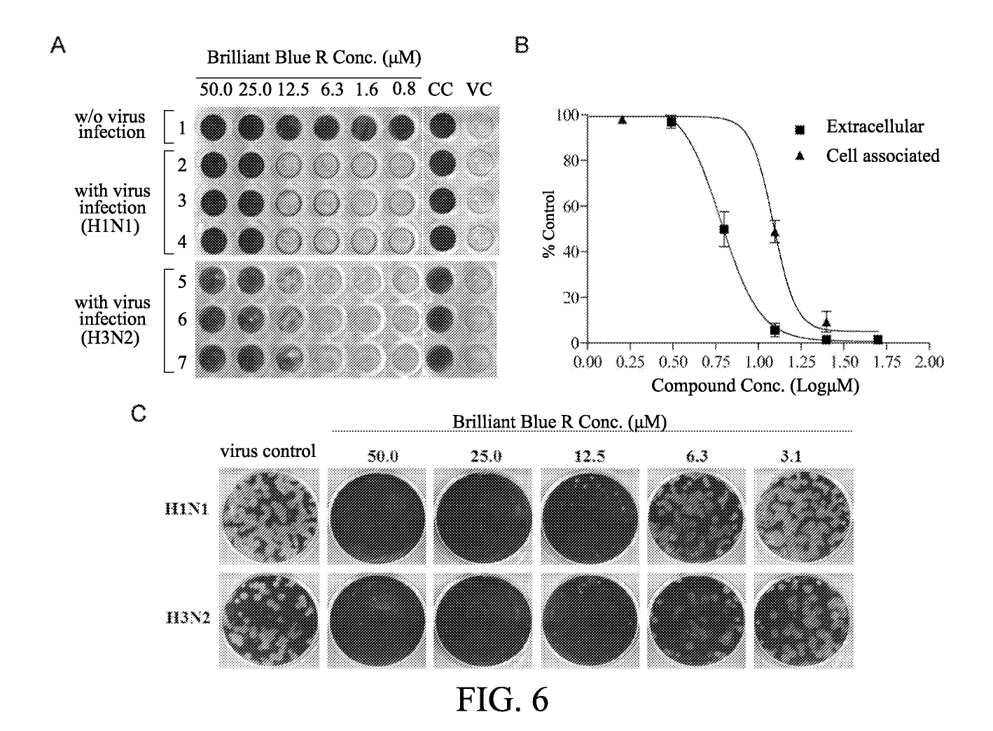

To examine if RB19 inhibits influenza virus replication, plaque-reduction assays were conducted. A/WSN/33 (H1N1) and A/Udorn/72 (H3N2) strains instead of the NIBRG14 (H5N1) strain for verification were used. In the plaque reduction assay, the low MOI was used to yield approximately 50-100 plaque forming units (PFU) per well of cells. Evaluation of RB19 at various concentrations in virus plaque reduction assays was performed for the two different influenza strains. In these experiments, RB19 reduced plaque forming units caused by infection of MDCK cells in a dose-dependent manner (FIGS. 6A and 6B). The EC₅₀ for viral plaque formation were estimated to be 2.7 μM and 2.8 μM for influenza A/WSN/33 (H1N1) and A/Udorn/72 (H3N2), respectively.

The effects of RB19 on the yields of viral progeny were also examined. Cultured MDCK cells were infected with A/WSN/33 at an MOI of 0.001, followed by the addition of RB19 at various concentrations. The culture supernatants and cell lysates were collected 48 hour after infection. Virus titers reflected the NA activity present in the collected samples. Treatment of cells with RB19 dramatically reduced virus yields, as indicated by the fluorogenic signal of NA activity (FIG. 6C). Furthermore, these results demonstrated that higher NA activity was present in cell lysates than in supernatants from RB19-treated infected cell cultures. These results indicated that RB19 treatment may result in the inhibition of influenza virions being released into the medium, thus indicating that NA activity was the major target of this novel anti-influenza compound.

Since RB19 was potent in enzyme- and cell-based assays, the anti-influenza activity of RB19 was also examined using two recent clinical isolates, A/TW/70058/09 (H1N1) and A/TW/70066/09 (H1N1), which were resistant to GS4071. Data from the viral plaque reduction assay showed that the EC₅₀ of RB19 on the inhibition of Influenza A/TW/70058/09 (H1N1) and Influenza A/TW/70066/09 (H1N1) replication were 9.8 μM and 11.9 μM, respectively. It was likely that the current drug-resistant problems may be alleviated by RB19 and/or its analogues.

TABLE 2

| | IC₅₀ (nM) (Fold change)^{b} | | |
|---|---|---|---|
| Type of Neuraminidase^{a} (NA) | 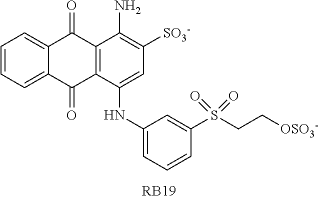 | 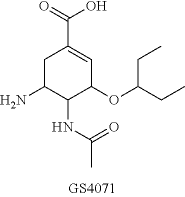 [SMILES: CCC(CC)OC1C=C(C(=O)O)CC(N)C1NC(C)=O] | 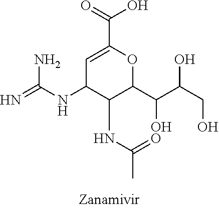 [SMILES: CC(=O)NC1C(NC(=N)N)C=C(C(=O)O)OC1C(O)C(O)CO] |
| NA^{WT} | 5.70 × 10³ | 0.13 | 0.65 |
| Bac-NA^{WT} | 3.4 × 10³(1) | 0.26 (1) | 0.54 (1) |
| Bac-NA^{I223R} | 3.0 × 10³(1) | 8.22(32) | 4.28(8) |
| Bac-NA^{H275Y} | 4.0 × 10³(1.2) | 417.2(1,604) | 1.26(2) |
| Bac-NA^{H275Y&I223R} | 4.5 × 10³(1.3) | 7,777.95(29,915) | 10.7(20) |

^{a}Neuraminidase (NA) had different mutations. The Bac-NA^{WT}, Bac-NA^{I223R}, Bac-NA^{H275Y} and Bac-NA^{H275Y&}1223R mutations were derived from the expression of NIBRG14 (H5N1) wild type, H275Y, I223R, and H275Y & I223R, respectively.
^{b}Fold change was obtained by comparing the IC₅₀ of mutant type and Bac-NA^{WT}.

Drug-resistant mutations often emerge during drug therapy and result in treatment failure. For example, the dual mutation of NA led to reduced susceptibility to NA-inhibiting drugs, which were detected in patients after treatment with oseltamivir and zanamivir. In another example, gefitinib and erlotinib are often used to target epidermal growth factor receptor for treating non-small-cell lung cancer. Although the drugs are effective, many patients eventually relapse due to resistance mutations. These examples demonstrated that better drug screening method is needed for screening drugs that are simultaneously effective against mutant type targets and drug-resistant type targets, and wherein the screening method is a novel method to screen out compounds for drug resistant type targets.

The novel drug screening method of the present invention was able to screen out compounds that can simultaneously inhibit wild type and multiple drug-resistant NAs.

The drug screening method herein described screened out the compound of formula (I), and in particular, RB19 was screened and found to be a potential drug candidate that can be an effective inhibitor of NA. Moreover, this inhibitor can also be used for the prevention or treatment of related diseases or disorders. The compound of formula (I) can also be useful starting points for designing effective NA inhibitors that can combat influenza viruses (virus containing H275Y mutation) resistant to oseltamivir, influenza viruses (containing I223R mutation) that are resistant to zanamivir, as well as influenza viruses (containing the I223R and H275Y mutations) that are multiple drug-resistant.

The foregoing descriptions are only illustrative of the features and functions of the present invention but are not intended to restrict the scope of the present invention. It is apparent to those skilled in the art that all equivalent modifications and variations made in the foregoing descriptions according to the spirit and principle in the disclosure of the present invention should fall within the scope of the appended claims.

What is claimed is:

1. A drug screening method, comprising steps of:
screening a compound library to obtain a first group of compounds capable of binding to a wild type target;
screening the first group of compounds to obtain a second group of compounds capable of binding to a mutant site of a mutant target;
analyzing characteristics of binding sites of the wild type target and the mutant type target to obtain physico-chemical properties of the binding sites;
identifying a candidate from the second group of compounds according to the physico-chemical properties of the binding site; and
performing a bio-assay on inhibitory activity of the candidate.

2. The drug screening method of claim 1, wherein the first group of compounds are screened out from the compound library by a molecular docking tool, and the second group of compound are screened out from the first group of compounds by the molecular docking tool.

3. The drug screening method of claim 2, wherein the molecular docking tool is GEMDOCK.

4. The drug screening method of claim 1, wherein the physico-chemical properties comprise an interaction between the second group of compounds and the binding sites.

5. The drug screening method of claim 1, wherein the candidate are capable of binding to the wild type target and mutant type target.

6. The drug screening method of claim 1, wherein the candidate is capable of interacting with the mutant site of the mutant type target.

7. The drug screening method of claim 1, wherein the candidate has an inhibitory potential on the wild type target and the mutant type target.

8. The drug screening method of claim 1, wherein physico-chemical properties of the candidate are complementary to the physico-chemical properties of the binding sites.

9. The drug screening method of claim 1, wherein the characteristics of binding sites of the wild type target and the mutant type target are demonstrated by a site-moiety map.

10. The drug screening method of claim 2, wherein docking models of the second group of compounds and the binding sites are used for analyzing the characteristics of the binding sites and establishing the site moiety map.

11. The drug screening method of claim 1, wherein the wild type target and the mutant type target are enzymes.

12. The drug screening method of claim 1, wherein the mutant type target is from a subject having a drug resistance.

13. The drug screening method of claim 1, wherein the wild type target and the mutant target are neuraminidases.

14. The drug screening method of claim 13, wherein the candidate is a potential inhibitor of neuraminidase.

15. The drug screening method of claim 13, wherein the candidate is used for treating diseases or disorders associated with neuraminidases.

16. The drug screening method of claim 15, wherein the candidate is used for treating influenza.

17. A method of inhibiting neuraminidase, comprising a step of administering to an subject a therapeutically effective amount of a compound of formula (I), 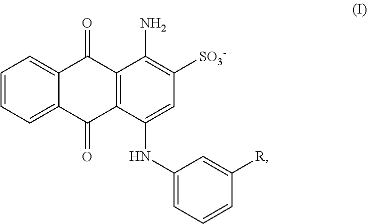
wherein R is one selected from the group consisting of:

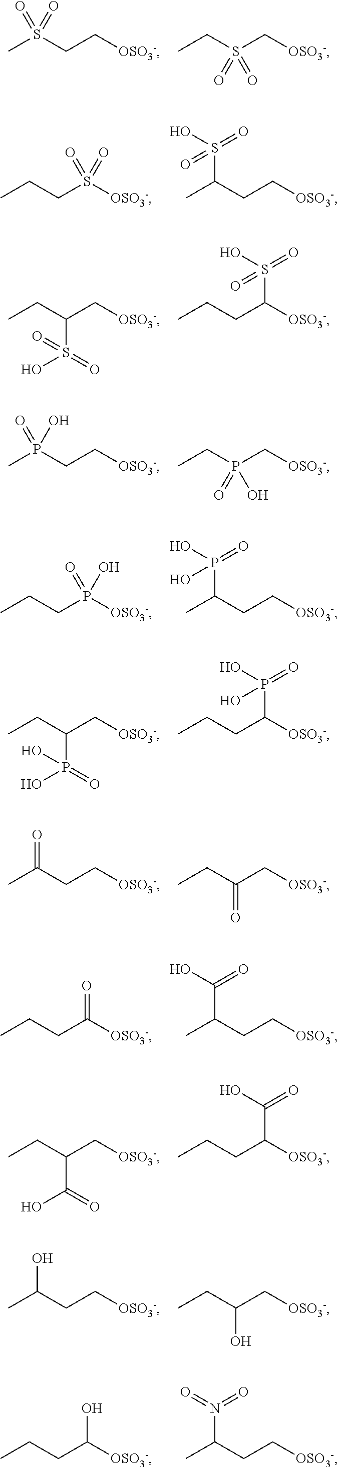

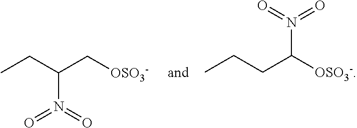

18. The method of claim 17, wherein the neuraminidase is a wild type neuraminidase or a mutant type neuraminidase.

19. The method of claim 17, wherein R is

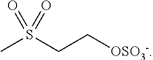

20. A method of treating influenza, comprising a step of administering to a subject a therapeutically effective amount of a compound of formula (I) 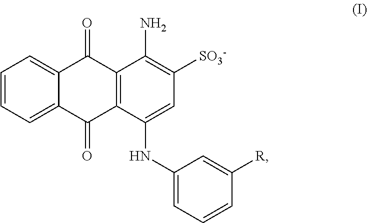
wherein R is one selected from the group consisting of:

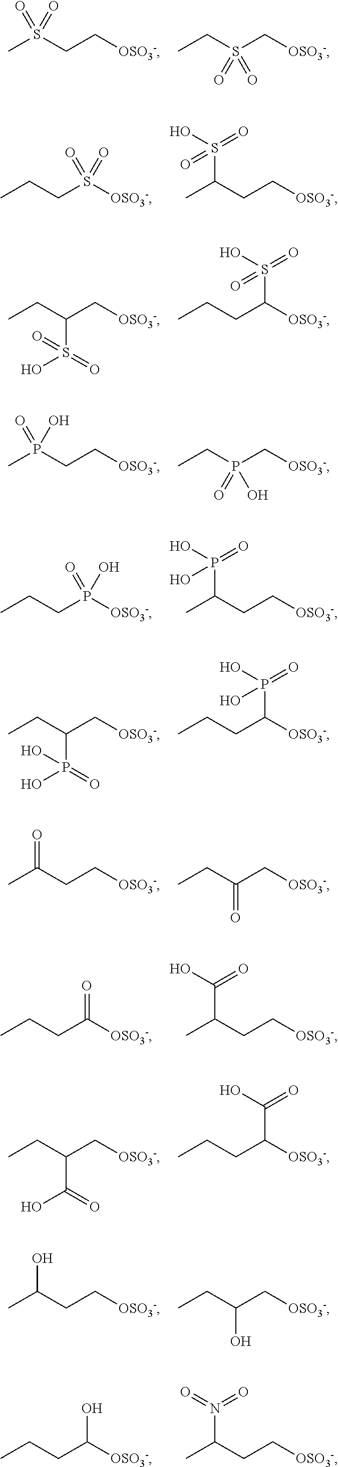

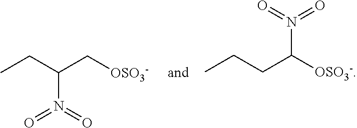

21. The method of claim 20, wherein the compound of formula (I) has inhibitory activity on neuraminidase, and the neuraminidase is a wild type neuraminidase or a mutant type neuraminidase.

22. The method of claim 20, wherein R is [chemical structure]


SEQUENCE LISTING (2 sequences)

SEQ ID NO: 1 - DNA, 38 bp, artificial sequence
  PCR primer
gagttggaggaacaacagactgagaactcaagagtctg

SEQ ID NO: 2 - DNA, 37 bp, artificial sequence
  PCR primer
cagctcttgagttctcagtctgttgttcctccaactc